(19) United States
(12) Patent Application Publication
(10) Pub. No.: US 2011/0106098 A1
(43) Pub. Date: May 5, 2011

(54) OCCLUSIVE DEVICE DELIVERY SYSTEM

(75) Inventor: Michael D Williams, Dover, NH (US)

(73) Assignee: Boston Scientific Scimed, Inc., Maple Grove, MN (US)

(21) Appl. No.: 12/913,085

(22) Filed: Oct. 27, 2010

Related U.S. Application Data
(60) Provisional application No. 61/257,377, filed on Nov. 2, 2009.

(51) Int. Cl. A61B 17/00

(52) U.S. Cl. 606/108

(57) ABSTRACT

A system for delivering an occlusive device includes a delivery wire assembly configured to be slidably inserted into and through the lumen of a delivery catheter, the delivery wire assembly including a delivery wire conduit defining a conduit lumen and having a conduit distal end forming a first cathode of a position detection circuit, a noble wire disposed in the conduit lumen and having a noble wire distal end forming an anode of the position detection circuit, and a core wire disposed in the conduit lumen and having a core wire distal end, the core wire distal end forming a second cathode of the position detection circuit, wherein an occlusive device is detachably coupled to the core wire distal end.

### RELATED APPLICATION DATA

The present application claims the benefit under 35 U.S.C. §119 to U.S. provisional patent application Ser. No. 61/257,377, filed Nov. 2, 2009. The foregoing application is hereby incorporated by reference into the present application in its entirety.

### FIELD OF THE INVENTION

The field of the disclosed inventions generally relates to systems and delivery devices for implanting vaso-occlusive devices for establishing an embolus or vascular occlusion in a vessel of a human or veterinary patient. More particularly, the disclosed inventions are directed to a system for detecting a position of a delivery wire assembly relative to a delivery catheter in an occlusive device delivery system.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The relative stiffness of the coil will depend, among other things, on its composition, the diameter of the wire strand, the diameter of the primary mandrel, and the pitch of the resulting primary windings. The coil is then wrapped around a larger, “secondary” mandrel, and heat treated to impart a secondary shape. For example, U.S. Pat. No. 4,994,069, issued to Ritchart et al., describes a vaso-occlusive coil that assumes a linear, helical primary shape when stretched for placement through the lumen of a delivery catheter, and a folded, convoluted secondary shape when released from the delivery catheter and deposited in the vasculature.

In order to deliver the vaso-occlusive devices to a desired site in the vasculature, e.g., within an aneurismal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” wire is then passed through the micro-catheter, until a vaso-occlusive device coupled to a distal end of the delivery wire is extended out of the distal end opening of the micro-catheter and into the aneurysm. The vaso-occlusive device is then released or “detached” from the end delivery wire, and the delivery wire is withdrawn back through the catheter. Depending on the particular needs of the patient, one or more additional occlusive devices may be pushed through the catheter and released at the same site.

One well-known way to release a vaso-occlusive device from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes an electrolytic detachment circuit with a return electrode, and the detachment zone disintegrates due to electrolysis.

In “monopolar” systems, return electrodes include electrodes attached to the patient's skin and conductive needles inserted through the skin at a remote site. In “bipolar” systems, return electrodes are located on the pusher wire but electrically insulated from the conductive path ending in the detachment zone.

The anode is made up of an insulated core wire, which runs through the pusher wire, is attached to an electrical contact at the proximal end, and forms the detachment zone at the distal end. The anode electrical contact is a metallic tube secured to the proximal end of the pusher wire.

The detachment performance of electrolytically severable junctions is subject to the availability of a clear electrical path between the anode and the cathode. If current is applied to the electrolytic detachment circuit while the detachment zone is inside of the catheter, the presence of the electrically insulative catheter reduces the available electrolytic pathway, and thus increases the impedance of the system, causing an increase in detachment time. To ensure proper alignment of the delivery wire and the catheter, radiopaque markers are placed on the delivery wire and the catheter. These markers are subject to mechanical tolerance stackup, so to ensure the detachment zone is sufficiently outside the catheter, the nominal position for the detachment zone using radiopaque marker alignment is just outside the catheter. An electrolytic detachment circuit can function properly with the detachment zone just barely outside or inside the catheter distal end.

United States Patent Application Publication No. 2005/0021023 discloses a system for positioning a detachment zone and an implant by monitoring a change in an electrical condition related to the position of the detachment zone in the catheter. The electrical condition (e.g., magnitude of alternating current) changes when the detachment zone exits the catheter and contacts a conductive component of the body, such as blood. In response to a change in the electrical condition, the system can signal a user or initiate detachment (e.g., by applying a direct current).

It is desirable that the detachment zone not be extended outside of the catheter any further than necessary. It is also desirable to avoid premature oxidation of the detachment zone when using electrolytic detachment due to the current used to detect position.

### SUMMARY

In one embodiment, a delivery wire assembly is provided for delivering an occlusive device to a location in a patient's vasculature. The delivery wire assembly includes a delivery wire conduit defining a conduit lumen and having a conduit distal end forming a first cathode of a position detection circuit, and a noble wire disposed in the conduit lumen and having a noble wire distal end forming an anode of the position detection circuit. The delivery wire assembly may further comprising a core wire disposed in the conduit lumen and having a core wire distal end, wherein the core wire distal end forms a second cathode of the position detection circuit. An occlusive device is detachably coupled to a distal end of the core wire.

In another embodiment, a system is provided for delivering an occlusive device to a location in a patient's vasculature, the system including a delivery catheter defining a catheter lumen, and a delivery wire assembly configured to be slidably inserted into and through the lumen of the delivery catheter, the delivery wire assembly comprising a delivery wire conduit defining a conduit lumen and having a conduit distal end, a noble metal plating disposed on the conduit distal end, and a core wire disposed in the conduit lumen and having a core wire distal end, wherein the noble metal plated conduit distal end forms an anode of a position detection circuit and the core wire distal end forms a cathode of the position detection circuit. An occlusive device is detachably coupled to the core wire distal end. A power supply is electrically connected to the delivery wire assembly, and a controller is electrically connected to the respective position detection circuit and the power supply, the controller configured to detect a change in an electrical characteristic of the position detection circuit, and to automatically cause the power supply to reverse the polarity of the position detection circuit when the change is detected. In various embodiments, the electrical characteristic is impedance between the anode and the cathode.

In another embodiment, a system includes a delivery catheter defining a catheter lumen, and a delivery wire assembly configured to be slidably inserted into and through the lumen of the delivery catheter, the delivery wire assembly comprising a delivery wire conduit defining a conduit lumen and having a conduit distal end forming a first cathode of a position detection circuit, a noble wire disposed in the conduit lumen and having a noble wire distal end forming an anode of the position detection circuit, and a core wire disposed in the conduit lumen and having a core wire distal end, wherein the core wire distal end forms a second cathode of the position detection circuit. The system further includes a controller configured to detect (i) a change in impedance between the anode and the first cathode of the position detection circuit, and (ii) a change in impedance between the anode and the second cathode of the position detection circuit, and to generate a respective signal when the respective change is detected.

These and other aspects and features of the disclosed inventions are described in the following detailed description, with reference to the accompanying figures.

### BRIEF DESCRIPTION OF THE DRAWINGS

Referring now to the drawings in which like reference numbers represent corresponding parts throughout, and in which:

FIG. 1 illustrates an occlusive coil delivery system, according to one embodiment.

FIG. 2 is a longitudinal cross-sectional view of a delivery wire assembly, according to the embodiment shown in FIG. 1.

FIG. 3 illustrates an occlusive coil in a natural state mode, illustrating one exemplary secondary configuration.

FIG. 4 is a longitudinal cross-sectional view of a delivery wire assembly, according to another embodiment.

FIG. 5 illustrates an occlusive coil delivery system, according to the embodiment shown in FIG. 4.

### DETAILED DESCRIPTION OF THE ILLUSTRATED EMBODIMENTS

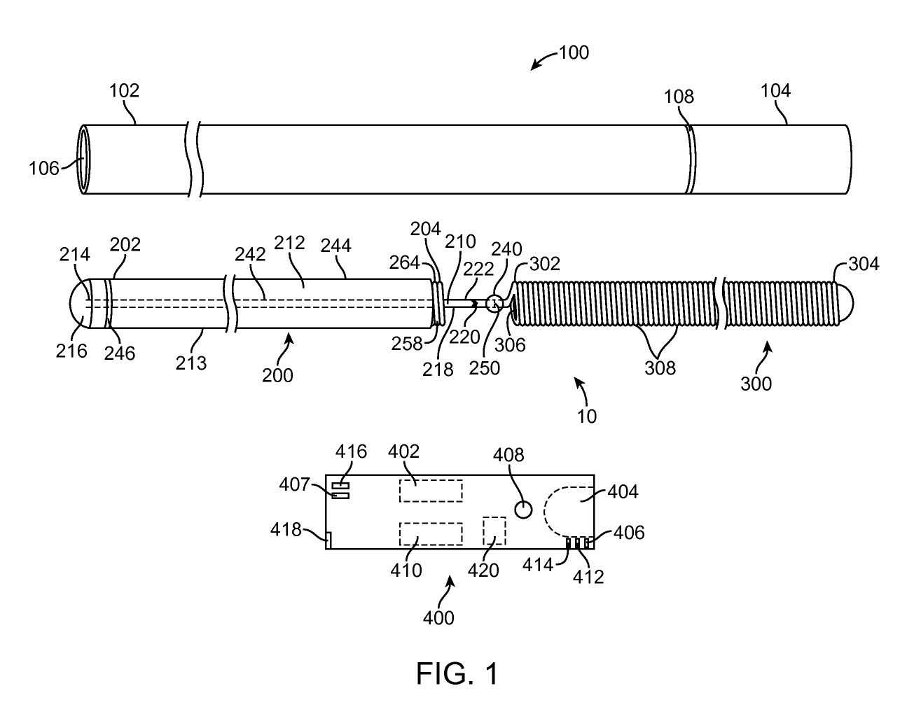

FIG. 1 illustrates an occlusive coil delivery system 10 according to one embodiment of the invention. The system 10 includes a number of subcomponents or sub-systems, including a delivery catheter 100, a delivery wire assembly 200, an occlusive coil 300, and a power supply 400. The delivery catheter 100 includes a proximal end 102, a distal end 104, and a lumen 106 extending between the proximal and distal ends 102, 104. The lumen 106 of the delivery catheter 100 is sized to accommodate axial movement of the delivery wire assembly 200. Further, the lumen 106 is sized for the passage of a guidewire (not shown), which may optionally be used to properly guide the delivery catheter 100 to the appropriate delivery site.

The delivery catheter 100 may include a braided-shaft construction of stainless steel flat wire that is encapsulated or surrounded by a polymer coating. By way of non-limiting example, HYDROLENE® is a polymer coating that may be used to cover the exterior portion of the delivery catheter 100. Of course, the system 10 is not limited to a particular construction or type of delivery catheter 100 and other constructions known to those skilled in the art may be used for the delivery catheter 100.

The inner lumen 106 is advantageously coated with a lubricious coating such as PTFE to reduce frictional forces between the delivery catheter 100 and the respective delivery wire assembly 200 and occlusive coil 300 being moved axially within the lumen 106. The delivery catheter 100 may include one or more optional marker bands 108 formed from a radiopaque material that can be used to identify the location of the delivery catheter 100 within the patient's vasculature system using imaging technology (e.g., fluoroscope imaging). The length of the delivery catheter 100 may vary depending on the particular application, but generally is around 150 cm in length. Of course, other lengths of the delivery catheter 100 may be used with the system 10 described herein.

The delivery catheter 100 may include a distal end 104 that is straight as illustrated in FIG. 1. Alternatively, the distal end 104 may be pre-shaped into a specific geometry or orientation. For example, the distal end 104 may be shaped into a “C” shape, an “S” shape, a “J” shape, a 45° bend, a 90° bend. The size of the lumen 106 may vary depending on the size of the respective delivery wire assembly 200 and occlusive coil 300, but generally the diameter of the lumen 106 of the delivery catheter 100 (I.D. of delivery catheter 100) is less than about 0.02 inches. The delivery catheter 100 is known to those skilled in the art as a microcatheter. While not illustrated in FIG. 1, the delivery catheter 100 may be utilized with a separate guide catheter (not shown) that aids in guiding the delivery catheter 100 to the appropriate location within the patient's vasculature.

Still referring to FIG. 1, the system 10 includes a delivery wire assembly 200 configured for axial movement within the lumen 106 of the delivery catheter 100. The delivery wire assembly 200 generally includes a proximal end 202 and a distal end 204. The delivery wire assembly 200 includes a delivery wire conduit 213, which has a proximal tubular portion 206 and a distal coil portion 208. The proximal tubular portion 206 may be formed from, for example, a flexible stainless steel hypotube. The distal coil portion 208 may be formed from, for example, stainless steel wire. The distal coil portion 208 may be bonded to the proximal tubular portion 206 in an end-to-end arrangement.

The delivery wire assembly 200 further includes a core wire 210 that extends from the proximal end 202 of the delivery wire assembly 200 to a location that is distal with respect to the distal end 204 of the delivery wire assembly 200. The core wire 210 is disposed within a conduit lumen 212 that extends within an interior portion of the delivery wire conduit 213. The distal end of the conduit lumen 212 is sealed with a centering/stopper coil 252, which is attached to the inside of the delivery wire conduit 213 by adhesive 240. The core wire 210 is formed from an electrically conductive material such as stainless steel wire. The proximal end 214 of the core wire 210 (shown in phantom) is electrically coupled to a core wire electrical contact 216 located at the proximal end 202 of the delivery wire assembly 200. The core wire electrical contact 216 is configured to interface with a corresponding electrical contact (not shown) in the power supply 400.

A portion of the core wire 210 is advantageously coated with an insulative coating 218. The insulative coating 218 may include polyimide. The entire length of the core wire 210 is coated with an insulative coating 218, except for the proximal end 214 of the core wire 210 that contacts the core wire electrical contact 216, and a small region 220 located in a portion of the core wire 210 that extends distally with respect to the distal end 204 of the delivery wire assembly 200. This latter, “bare” portion of the core wire 210 of the electrolytic detachment zone 220, which dissolves upon application of electrical current from the power supply 400 in the electrolytic detachment mode with the electrolytic detachment zone 220 configured as the anode.

Still referring to FIG. 1, the occlusive coil 300 includes a proximal end 302, a distal end 304, and a lumen 306 extending there between. The occlusive coil 300 is generally made from a biocompatible metal such as platinum or a platinum alloy (e.g., platinum-tungsten alloy). The occlusive coil 300 generally includes a straight configuration (as illustrated in FIG. 1) when the occlusive coil 300 is loaded within the delivery catheter 100. Upon release, the occlusive coil 300 generally takes a secondary shape which may include two-dimensional or three-dimensional configurations such as that illustrated in FIG. 3. The occlusive coil 300 includes a plurality of coil windings 308. The coil windings 308 are generally helical about a central axis disposed along the lumen 306 of the occlusive coil 300. The occlusive coil 300 may have a closed pitch configuration as illustrated in FIG. 1. Of course, the system 10 described herein may be used with occlusive coils 300 or other occlusive structures having a variety of configurations, and is not limited to occlusive coils 300 having a certain size or configuration.

The distal end 222 of the core wire 210, which includes the electrolytic detachment zone 220, is connected to the proximal end 302 of the occlusive coil 300 at a junction 250. Various techniques and devices can be used to connect the core wire 210 to the occlusive coil 300, including laser melting, and laser tack, spot, and continuous welding. It is preferable to apply an adhesive 240 to cover the junction 250 formed between the distal end 222 of the core wire 210 and the proximal end 302 of the occlusion coil 300. The adhesive 240 may include an epoxy material which is cured or hardened through the application of heat or UV radiation. For example, the adhesive 240 may include a thermally cured, two-part epoxy such as EPO-TEK® 353ND-4 available from Epoxy Technology, Inc., 14 Fortune Drive, Billerica, Mass. The adhesive 240 encapsulates the junction 250 and increases its mechanical stability.

Still referring to FIG. 1 the system 10 further includes a power supply 400 for supplying direct current to the core wire 210, which ends distally in the electrolytic detachment zone 220. In the presence of an electrically conductive fluid (including a physiological fluid such as blood, or an electrically conductive flushing solution such as saline), activation of the power supply 400, in the electrolytic detachment mode, causes electrical current to flow in a circuit including the core wire electrical contact 216, the core wire 210, the electrolytic detachment zone 220, and a return electrode. In this circuit, the electrolytic detachment zone 220 is configured as the anode and the return electrode is configured as the cathode. After several seconds (generally less than about 10 seconds), the sacrificial electrolytic detachment zone 220 dissolves, and the occlusive coil 300 separates form the core wire 210.

The power supply 400 preferably includes an onboard energy source, such as batteries (e.g., a pair of AAA batteries), along with drive circuitry 402. The drive circuitry 402 may include one or more microcontrollers or processors configured to output a driving current. The power supply 400 illustrated in FIG. 1 includes a receptacle 404 configured to receive and mate with the proximal end 202 of the delivery wire assembly 200. Upon insertion of the proximal end 202 into the receptacle 404, the core wire electrical contact 216 disposed on the delivery wire assembly 200 electrically couple with corresponding contacts (not shown) located in the power supply 400.

A visual indicator 406 (e.g., LED light) is used to indicate when the proximal end 202 of delivery wire assembly 200 has been properly inserted into the power supply 400. Another visual indicator 407 is activated if the onboard energy source needs to be recharged or replaced. The power supply 400 includes an activation trigger or button 408 that is depressed by the user to apply the electrical current to the sacrificial electrolytic detachment zone 220. Once the activation trigger 408 has been activated, the driver circuitry 402 automatically supplies current until detachment occurs. The drive circuitry 402 typically operates by applying a substantially constant current, e.g., around 1.5 mA.

The power supply 400 may include optional detection circuitry 410 that is configured to detect when the occlusive coil 300 has detached from the core wire 210. The detection circuitry 410 may identify detachment based upon a measured impedance value. A visual indicator 412 may indicate when the power supply 400 is being supplied to the current to the sacrificial electrolytic detachment zone 220. Another visual indicator 414 may indicate when the occlusive coil 300 has detached from the core wire 210. As an alternative to the visual indicator 414, an audible signal (e.g., beep) or even tactile signal (e.g., vibration or buzzer) may be triggered upon detachment. The detection circuitry 410 may be configured to disable the drive circuitry 402 upon sensing detachment of the occlusive coil 300.

The power supply 400 may also contain another visual indicator 416 that indicates to the operator when non-bipolar delivery wire assembly is inserted into the power supply 400. Non-bipolar delivery wire assemblies use a separate return electrode that typically is in the form of a needle that was inserted into the groin area of the patient. The power supply 400 is configured to detect when a non-bipolar delivery wire assembly has been inserted. Under such situations, the visual indicator 416 (e.g., LED) is turned on and the user is advised to insert the separate return electrode (not shown in FIG. 1) into a port 418 located on the power supply 400.

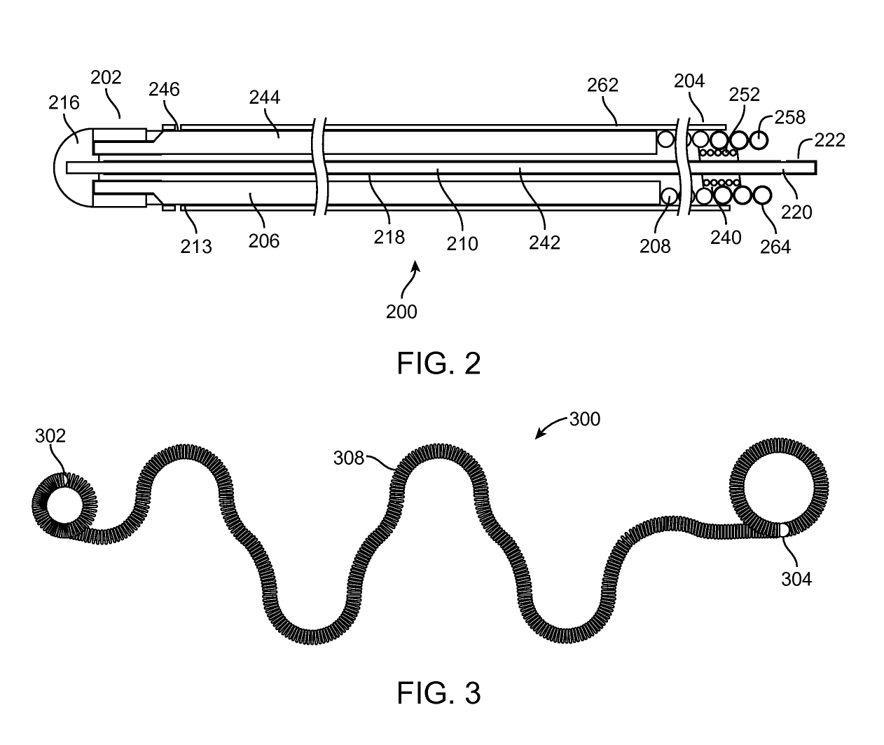

Referring to FIG. 2, the core wire 210 forms a first conductive path 242 between the core wire electrical contact 216 and the electrolytic detachment zone 220. In the electrolytic detachment mode, this first conductive path 242 comprises the anode (+) of the electrolytic detachment circuit when the delivery wire assembly 200 is operatively coupled to the power supply 400. A second conductive path 244, the return path, is formed by the proximal tubular portion 206 and a distal coil portion 208 of the delivery wire conduit 213. The second conductive path 244 is electrically isolated from the first conductive path 242. In the electrolytic detachment mode, the second conductive path 244 comprises the cathode (−) or ground electrode for the electrolytic detachment circuit.

A delivery wire conduit electrical contact 246 for the second conductive path 244 may be disposed on a proximal end of the tubular portion 206 of the delivery wire conduit 213. In one embodiment, the delivery wire conduit electrical contact 246 is simply an exposed portion of the tubular portion 206 since the tubular portion 206 is part of the second conductive path 244. For instance, a proximal portion of the tubular portion 206 that is adjacent to the core wire electrical contact 216 may be covered with an insulative coating 207 such as polyimide as illustrated in FIG. 2. An exposed region of the tubular portion 206 that does not have the insulative coating may form the delivery wire conduit electrical contact 246. Alternatively, the delivery wire conduit electrical contact 246 may be a ring type electrode or other contact that is formed on the exterior of the tubular portion 206.

The delivery wire conduit electrical contact 246 is configured to interface with a corresponding electrical contact (not shown) in the power supply 400 when the proximal end 202 of the delivery wire assembly 200 is inserted into the power supply 400. The delivery wire conduit electrical contact 246 of the second conductive path 244 is, of course, electrically isolated with respect to the core wire electrical contact 216 of the first conductive path 242.

As shown in FIG. 2, the delivery wire assembly 200 forms a position detection circuit with the detachment zone 220 forming a cathode and the distal end 258 of the delivery wire conduit 213 forming an anode. The distal end 258 of the delivery wire conduit 213 is plated with a noble metal (e.g., gold or platinum). Noble metals are resistant to oxidation and the noble metal plating prevents the distal end 258 of the delivery wire conduit 213 from dissolving under the current applied during position detection. The controller 420 in the power source 400 configures the flow of electrons such that the detachment zone 220 forms the cathode and the distal end 258 of the delivery wire conduit 213 forms the anode of the position detection circuit.

The same parts of the delivery wire assembly 200 also form an electrolytic detachment circuit with the detachment zone 220 forming an anode and the distal end 258 of the delivery wire conduit 213 forming a cathode. Because the detachment zone 220 is bare stainless steel wire, it oxidizes and dissolves under the current applied during electrolysis. For the electrolytic detachment circuit, the controller 420 configures the flow of electrons such that the detachment zone 220 forms the anode and the distal end 258 of the delivery wire conduit 213 forms the cathode.

The polarity reversal during detection minimizes the risk of inadvertently dissolving the detachment zone 220 during detection. The anode of the position detection circuit (i.e., the distal end 258 of the delivery wire conduit 213) is large enough that it is not substantially oxidized even without a noble metal plating. With a noble metal plating, the distal end 258 of the delivery wire conduit 213 will not be oxidized at all.

Still referring to FIG. 2, an outer sleeve 262 or jacket surrounds a portion of the proximal tubular portion 206 and a portion of the distal coil portion 208 of the delivery wire conduit 213. The outer sleeve 262 covers the interface or joint formed between the proximal tubular portion 206 and the distal coil portion 208. The outer sleeve 262 may have a length of around 50 cm to around 54 cm. The outer sleeve 262 may be formed from a polyether block amide plastic material (e.g., PEBAX 7233 lamination). The outer sleeve 262 may include a lamination of PEBAX and HYDROLENE® that may be heat laminated to the delivery wire assembly 200. The OD of the outer sleeve 262 may be less than 0.02 inches and advantageously less than 0.015 inches.

The core wire 210, which runs through the delivery wire conduit 213, terminates at core wire electrical contact 216 at one end and extends distally with respect to the distal coil portion 208 of the delivery wire conduit 213 to the core wire distal end 222 at the other end. The core wire 210 is coated with an insulative coating 218 such as polyimide except at the electrolytic detachment zone 220 and the proximal segment coupled to the core wire electrical contact 216. The electrolytic detachment zone 220 is located a short distance (e.g., about 0.02 mm to about 0.2 mm) distally with respect to the distal end of the distal coil portion 208. The core wire 210 may have an OD of around 0.002 inches.

FIG. 3 illustrates one exemplary configuration of an occlusive coil 300 in a natural state. In the natural state, the occlusive coil 300 transforms from the straight configuration illustrated in, for instance, FIG. 1 into a secondary shape. The secondary shaped may include both two and three dimensional shapes of a wide variety. FIG. 3 is just one example of a secondary shape of an occlusive coil 300 and other shapes and configurations are contemplated to fall within the scope of the invention. Also, the occlusive coil 300 may incorporate synthetic fibers over all or a portion of the occlusive coil 300 as is known in the art. These fibers may be attached directly to coil windings 308 or the fibers may be integrated into the occlusive coil 300 using a weave or braided configuration.

In use, the catheter 100 is threaded (e.g., through a surgical incision) to the target. Then the distal end of the delivery wire assembly 200 and the occlusive device 300 releasably attached thereto are inserted into the proximal end 102 of the catheter 100. The delivery wire assembly 200 and the occlusive device 300 are inserted a predetermined distance that positions the detachment zone 220 about 5-10 mm short of the length of the catheter 100. Next, with the detachment zone 220 forming a cathode and the distal end 258 of the delivery wire conduit 213 forming an anode, the controller 420 measures a baseline electronic characteristic, such as impedance, current, or voltage. Then the delivery wire assembly 200 and the occlusive device 300 are advanced distally about 0.2-1.0 mm further into the catheter 100. The controller 420 takes a new measurement of the electronic characteristic and calculates at difference between the new measurement and the base measurement. Next, the controller 420 compares the difference to a predetermined value (e.g., 20,000 ohms). If the difference compared to the predetermined value indicates that the distal end 204 of the delivery wire assembly 200 has not exited the catheter 100 (e.g., high impedance), the delivery wire assembly 200 and the occlusive device 300 are again advanced into the catheter 100. Then the controller 420 takes another new measurement, calculates a new difference, and makes a new comparison. If the difference indicates that the distal end 204 of the delivery wire assembly 200 has exited the catheter 100 (e.g., low impedance), the controller 420 initiates the detachment of the occlusive device 300. Accuracy of electronic characteristic measurement improves with the amount of power applied to the position detection circuit. Thus, using a noble material for the anode of the position detection circuit allows for greater accuracy, without sacrificing mechanical stability, by enabling higher power usage without oxidation of the anode.

In other embodiments, when the delivery detachment zone 220 is about 5 mm to about 10 mm from the distal end 102 of the catheter 100, the controller 420 samples the electronic characteristic continuously, at about 4 to about 10 samples and difference calculations per second. In such embodiments, the advance distance between calculations is dependent on the user's advance speed. In still other embodiments, no baseline measurement is taken. The controller 420 compares the measured electronic characteristic, either after every advance or continuously as described above. The controller 420 compares each new measured electronic characteristic against a threshold value without taking a difference (i.e., an impedance of below 10 kohm).

The controller 420 initiates detachment of the occlusive device 300 by redirecting the flow of electrons so that the detachment zone 220 forms the anode and the distal end 258 of the delivery wire conduit 213 forms the cathode. Then the power supply 400 delivers a current to the electrolytic detachment circuit. The current oxidizes the bare stainless steel wire in the detachment zone 220, which releases the occlusive device 300.

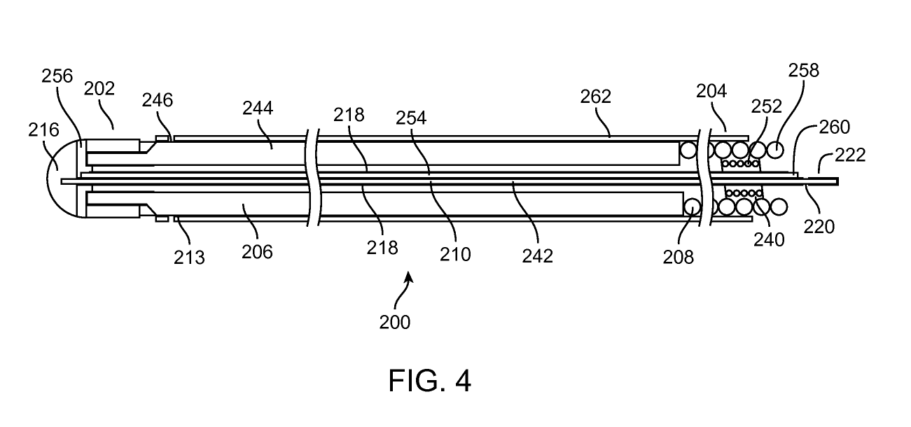

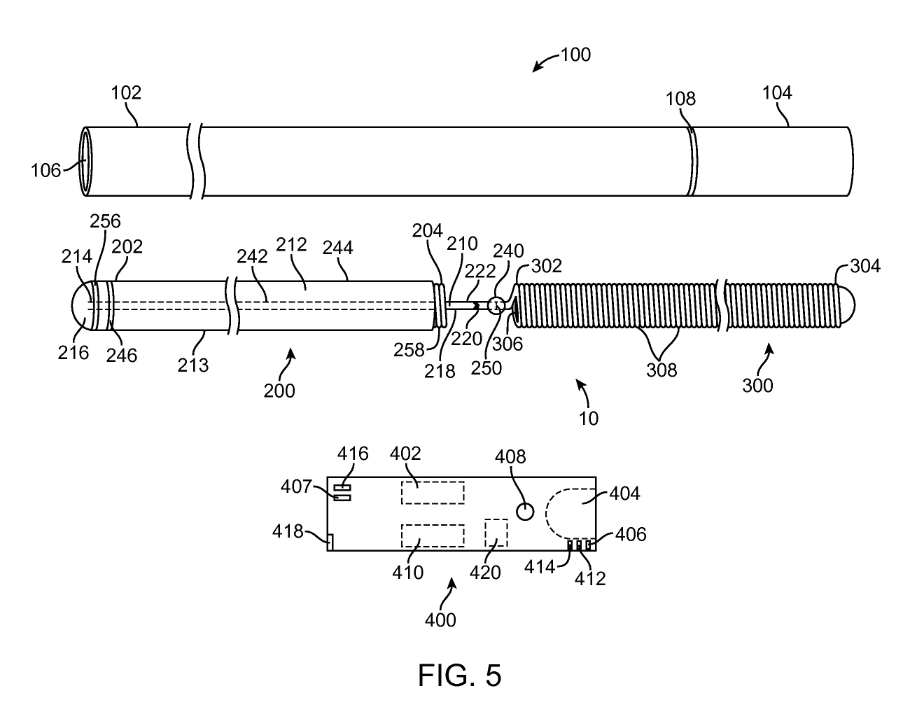

The embodiment depicted in FIGS. 4 and 5 is similar to the embodiment depicted in FIGS. 1 and 2, except that a noble wire 254 is also disposed in the delivery wire conduit 213 adjacent the core wire 210. The noble wire 254 and the core wire 210 are electrically isolated from each other by insulation 218. The noble wire 254 is insulated throughout its length with two exceptions. At the proximal end, the noble wire 254 is not insulated (i.e., bare) and it is connected to the noble wire electrode 256, which is configured to configured to interface with a corresponding electrical contact (not shown) in the power supply 400. At the distal end 260, the noble wire 254 is not insulated and forms the anode of the position detection circuit.

The distal end 258 of the delivery wire conduit 213 forms the first cathode of the position detection circuit. The detachment zone 220 forms the second cathode. In position detection mode, the controller 420 in the power supply 400 configures the flow of electrons so that distal end 260 of the noble wire 254 is the anode, and the distal end 258 of the delivery wire conduit 213 and the detachment zone 220 are the first and second cathodes. In electrolytic detachment mode, the controller 420 reconfigures the flow of electrons so the distal end 258 of the delivery wire conduit 213 is the cathode and the detachment zone 220 is the anode.

In use, this embodiment is similar to the embodiment in FIGS. 1 & 2. In addition to measuring one electronic characteristic, the controller 420 can measure a first and a second electronic characteristic (i.e., between the anode and the first cathode and between the anode and the second cathode). When the controller 420 determines that both the first and second electronic characteristics indicate that the distal end 258 of the delivery wire conduit 213 and the detachment zone 220 are both outside of the catheter 100, the controller 420 can initiate electrolytic detachment as described above.

While various embodiments of the present invention have been shown and described, they are presented for purposes of illustration, and not limitation. Various modifications may be made to the illustrated and described embodiments without departing from the scope of the present invention, which is to be limited and defined only by the following claims and their equivalents. For instance the above described systems and methods will indicate the position of any elongate body relative to a catheter. Further, the controller 420 can trigger any detachment mechanism.

What is claimed is:

1. A delivery wire assembly for delivering an occlusive device to a location in a patient's vasculature, comprising:
a delivery wire conduit defining a conduit lumen and having a conduit distal end forming a first cathode of a position detection circuit; and
a noble wire disposed in the conduit lumen and having a noble wire distal end forming an anode of the position detection circuit.

2. The delivery wire assembly of claim 1, further comprising a core wire disposed in the conduit lumen and having a core wire distal end, wherein the core wire distal end forms a second cathode of the position detection circuit.

3. A system for delivering an occlusive device to a location in a patient's vasculature, comprising:
a delivery catheter defining a catheter lumen;
a delivery wire assembly configured to be slidably inserted into and through the lumen of the delivery catheter, the delivery wire assembly comprising
a delivery wire conduit defining a conduit lumen and having a conduit distal end,
a noble metal plating disposed on the conduit distal end, and
a core wire disposed in the conduit lumen and having a core wire distal end, wherein the noble metal plated conduit distal end forms an anode of a position detection circuit and the core wire distal end forms a cathode of the position detection circuit;
an occlusive device detachably coupled to the core wire distal end;
a power supply electrically connected to the delivery wire assembly; and
a controller electrically connected to the respective position detection circuit and the power supply, wherein the controller is configured to detect a change in an electrical characteristic of the position detection circuit, and to automatically cause the power supply to reverse the polarity of the position detection circuit when the change is detected.

4. The system of claim 3, wherein the electrical characteristic is impedance between the anode and the cathode.

5. A system for delivering an occlusive device to a location in a patient's vasculature, comprising:
a delivery catheter defining a catheter lumen;
a delivery wire assembly configured to be slidably inserted into and through the lumen of the delivery catheter, the delivery wire assembly comprising
a delivery wire conduit defining a conduit lumen and having a conduit distal end forming a first cathode of a position detection circuit,
a noble wire disposed in the conduit lumen and having a noble wire distal end forming an anode of the position detection circuit, and
a core wire disposed in the conduit lumen and having a core wire distal end, wherein the core wire distal end forms a second cathode of the position detection circuit; and
a controller configured to detect a change in impedance between the anode and the first cathode of the position detection circuit, and to generate a signal when the change is detected.

6. The system of claim 5, the controller further configured to detect a change in impedance between the anode and the second cathode of the position detection circuit, and to generate a signal when the respective change is detected.
